(19) United States
(12) Patent Application Publication
(10) Pub. No.: US 2026/0139287 A1
(43) Pub. Date: May 21, 2026

(54) ANTIBIOTIC SUSCEPTIBILITY TESTING DEVICE AND METHOD THEREOF

(71) Applicant: BIOGUARD CORPORATION, New Taipei City (TW)

(72) Inventor: YU-CHEN CHOU, New Taipei City (TW)

(21) Appl. No.: 19/031,163

(22) Filed: Jan. 17, 2025

(30) Foreign Application Priority Data
Nov. 15, 2024 (TW) 113144087

(51) Int. Cl. C12Q 1/18; C12M 1/34

(52) U.S. Cl. CPC C12Q 1/18; C12M 41/12; C12M 41/36

(57) ABSTRACT

Disclosed are an antibiotic susceptibility testing device and a method thereof, in which before the testing device is activated to carry out testing, a growth recognition light source is operated to illuminate culture elements, and a light inspection module receives a growth message obtained from the number of the original specimen so as to determine if cultivation is completed. Then, the specimen is diluted and introduced into reaction receptacles of a reagent disc, of which the entirety is placed into an inspection setup tank to start the testing. After reaction, first and second inspection light sources of different wavelengths are sequentially applied to illuminate the same reaction receptacle. And, finally, a spectrum processing module converts a color message obtained from an AST-dedicated indicator into a wavelength message, to thereby obtain the result of inspection.

### BACKGROUND OF THE INVENTION

### (a) Technical Field of the Invention

The present invention provides an antibiotic susceptibility testing (AST) device, and a method thereof, for accurately determining a growth condition of specimen before and after the AST test and a result of inspection in a simple and fast manner.

### (b) Description of the Prior Art

Antibiotic susceptibility testing (AST), also known as antibiotic sensitivity testing, is a test designed to assess the sensitivity of bacteria, fungi, or other microorganisms to antibiotic drugs (such as antibiotics) in order to select the most effective treatment drugs.

Before and after the AST experiment, the concentration or amount of the specimen needs to be inspected. The inspection before the experiment is for the microbial culture status, and it is necessary to confirm that the microorganisms in the culture dish have reached a certain concentration before the number of specimens required for the subsequent experiment can be supplied. The inspection after experiment is for the status of reaction between microorganisms and antibiotics, and it is necessary to confirm the number of microorganisms after the reaction in order to determine whether the antibiotic meets the experimental requirements.

However, the specimens for the above-described AST experiment have the following problems and deficiencies when used, which need to be improved: Firstly, generally, to inspect the number of microorganisms in a culture dish, the microorganisms are usually grown for several or dozens of hours under specific temperature conditions, and the time of cultivation is used as the judgment that the microorganisms have reached a certain concentration. However, considering the growth status and conditions of microorganisms may vary, using such an imprecise way of culture to judge whether the microorganisms are growing normally, even with a timer reminder function, the results still need to be manually checked. If someone judges that the growth is not favorable, the culture dish will be repeatedly removed and re-placed in a space of conditions suitable for growth. This will lead to the risk of misjudgment, unknown specimen concentration or specimen volume, and risk of infection due to repeated removal and inspection.

Secondly, the results of traditional AST experiments are determined by the Kirby-Bauer disk diffusion method, and the sensitivity of the specimen to antibiotics is determined by measuring the size of the inhibition zone. This method has a high risk of misjudgment, and although methods that use darkness of the color of color indicators after reaction to make judgement are available, manual inspection is still necessary, and the accuracy and correctness of the experimental results are questionable.

### SUMMARY OF THE INVENTION

Thus, in view of the above problems, the present inventor has collected relevant information, evaluated and considered various aspects, and based on many years of experience in this industry, creates an antibiotic susceptibility testing device, and a method thereof, for accurately determining a growth condition of specimen before and after the AST test and a result of inspection in a simple and fast manner.

The main purpose of the present invention is to use an arrangement of a growth recognition light source and a light inspection module to accurately determine a growth status of specimen in a culture element in order to eliminate manual interpretation and reduce risk of infection, and also to simultaneously learn the concentration of the specimen after growth.

Another main purpose of the present invention is to use an arrangement of a first inspection light source, a second inspection light source, and a spectrum processing module to perform, in sequence, illumination with different wavelengths for cross-checking and to convert a color message obtained with an AST-dedicated indicator into a wavelength message for acquiring the result of inspection fast and accurately.

To achieve the above purposes, a structure of the present invention includes: a testing device, at least two inspection setup tanks, a reagent disc, a plurality of reaction receptacles for receiving and holding an AST-dedicated indicator, at least two culture elements for receiving an original specimen, a growth recognition light source, at least two first inspection light sources, at least two second inspection light sources having wavelength different from the first inspection light sources, at least one light inspection module, and at least one spectrum processing module. The inspection setup tanks are set in the testing device. The reaction receptacles are arranged in the reagent disc. The culture elements are respectively arranged at one side of the inspection setup tanks. The growth recognition light source is arranged at one side of the culture elements to illuminate the original specimen. The first inspection light sources are arranged at one side of the inspection setup tanks to illuminate any one of the reaction receptacles. The second inspection light sources are arranged at one side of the inspection setup tanks and illuminate the same reaction receptacles with the first inspection light sources. The light inspection module acquires a growth message from a number of the original specimens obtained by illuminating the culture elements with the growth recognition light source. The spectrum processing module receives a color message obtained with the AST-dedicated indicator by illuminating the reaction receptacles with the first inspection light sources and the second inspection light sources for conversion into a wavelength message.

Before using the present invention to perform antibiotic susceptibility testing, a user first places the original specimen in the culture element inside the testing device for pre-cultivation, and during the cultivation period, the growth recognition light source can be used to illuminate the culture element, and after the light inspection module receives the illumination of the growth recognition light source, the growth message obtained from the change of the number of the original specimen is used to determine whether it has been cultivated into a usable specimen. Then, the usable specimen is diluted to become the appropriate sample, and added to the reaction receptacles of the reagent disc, and then placed in the inspection setup tanks of the testing device to then activate the testing device. After the reaction is completed, the first inspection light sources and the second inspection light sources of different wavelengths are sequentially applied to illuminate the appropriate specimen in the reaction receptacles. Finally, the spectrum processing module is operated to receive the color message obtained with illumination of the AST-dedicated indicator in the reaction receptacles for conversion into the wavelength message, to thereby obtain the result of inspection. As such, the growth status of specimen and the result of inspection can be accurately determined in a simple and fast manner, and the result of inspection is analyzed with two inspection light sources and the spectrum processing module, so as to eliminate manual interpretation and to obtain a more accurate result through cross-checking.

Through the above-described technology, the problems of inspecting specimen in the prior art AST experiments with respect to error of manual interpretation, unknown specimen concentration and specimen volume, easy infection resulting from multiple times of removal and inspection, and poor accuracy and correctness due to all requiring manual inspection of experiment results can be overcome to achieve the practical progress mentioned above.

### BRIEF DESCRIPTION OF THE DRAWINGS

FIG. 1 is a perspective see-through view showing a preferred embodiment of the present invention in an expanded state.

FIG. 2 is an exploded view showing the preferred embodiment of the present invention.

FIG. 3 is a cross-sectional view showing the preferred embodiment of the present invention.

FIG. 4 is a flow chart showing steps of the preferred embodiment of the present invention.

FIG. 5 is a schematic view showing culture elements of the preferred embodiment of the present invention.

FIG. 6 is a schematic view illustrating specimen introducing according to the preferred embodiment of the present invention.

FIG. 7 is a schematic view illustrating reaction receptacle inspecting according to the preferred embodiment of the present invention.

FIG. 8 is a schematic view illustrating specimen recognizing according to another preferred embodiment of the present invention.

FIG. 9 is a schematic view illustrating displaying according to said another preferred embodiment of the present invention.

FIG. 10 is a schematic view illustrating temperature uniformizing according to a further preferred embodiment of the present invention.

FIG. 11 is a schematic view illustrating light converging according to said further preferred embodiment of the present invention.

### DETAILED DESCRIPTION OF THE PREFERRED EMBODIMENTS

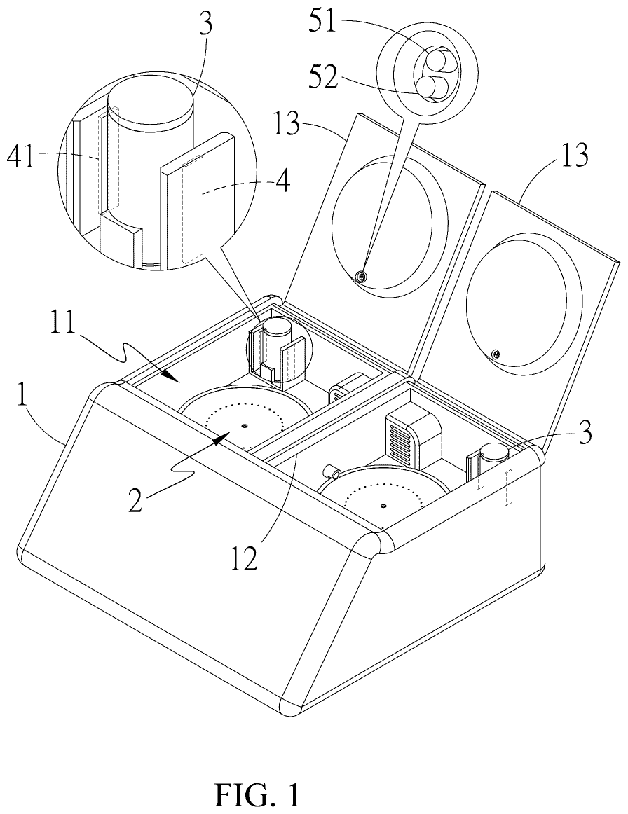

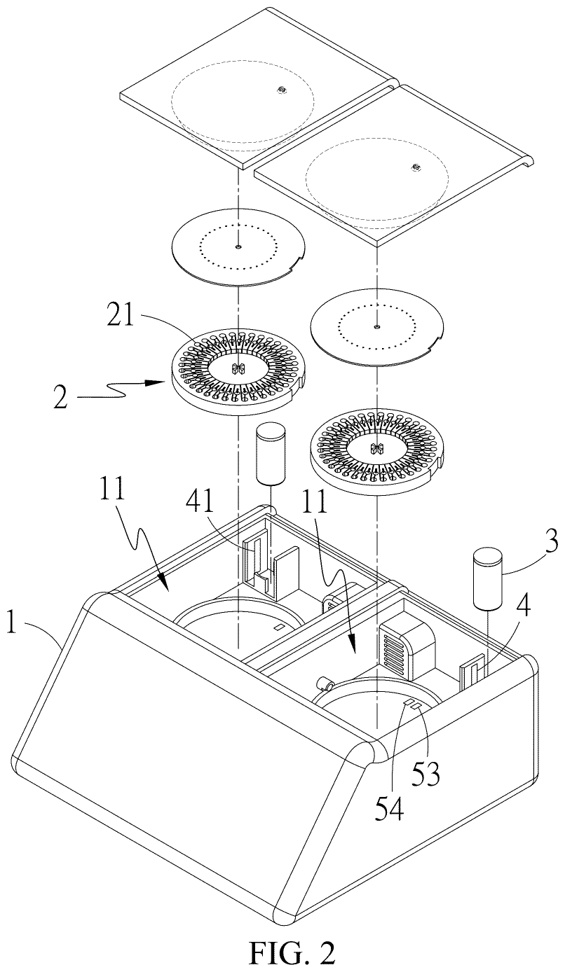

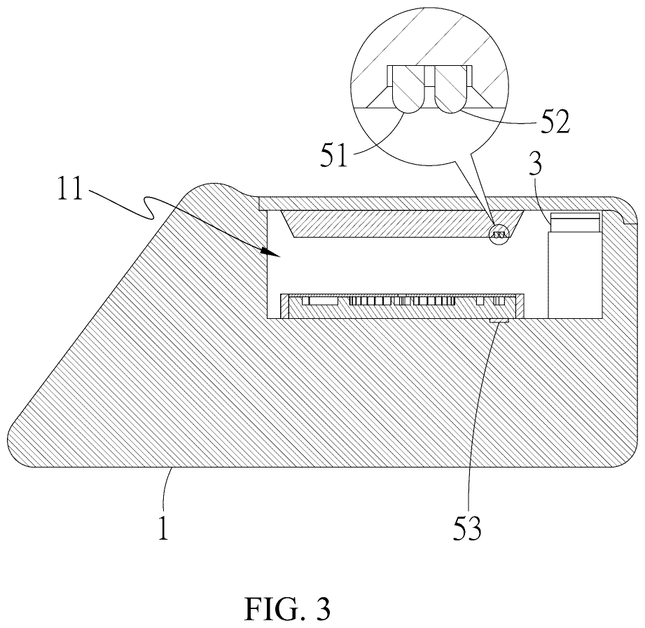

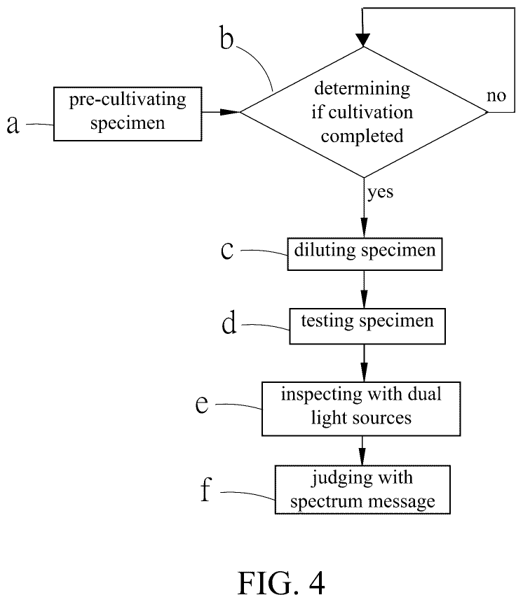

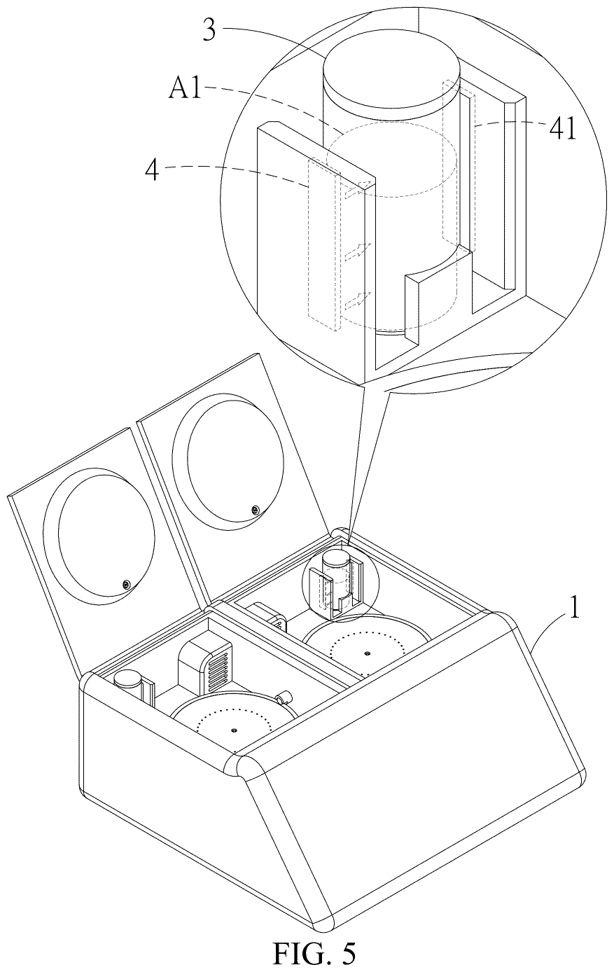

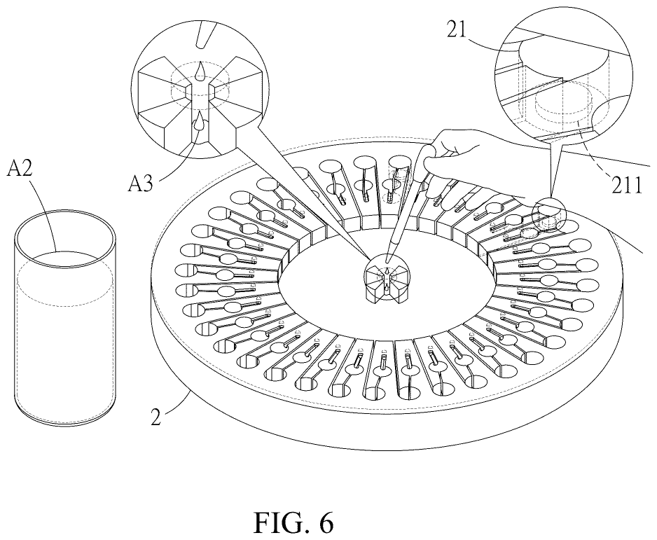

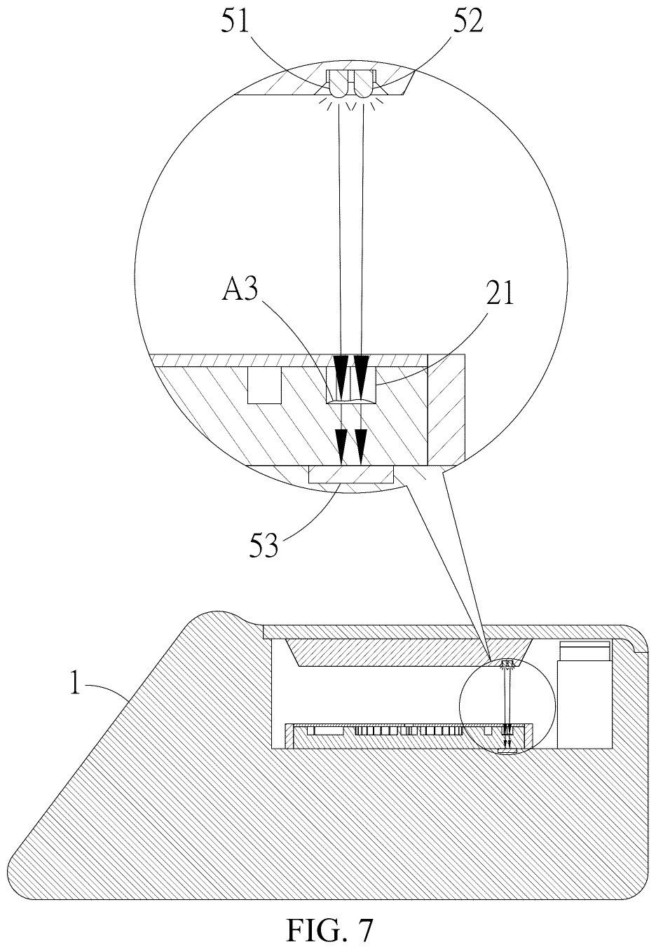

Referring to FIGS. 1-7, which are respectively a perspective see-through view showing a preferred embodiment of the present invention in an expanded state, an exploded view showing the preferred embodiment of the present invention, a cross-sectional view showing the preferred embodiment of the present invention, a flow chart showing steps of the preferred embodiment of the present invention, a schematic view showing culture elements of the preferred embodiment of the present invention, a schematic view illustrating specimen introducing according to the preferred embodiment of the present invention, and a schematic view illustrating reaction receptacle inspecting according to the preferred embodiment of the present invention, it is clearly seen from the drawings that the present invention comprises:
a testing device 1;
at least two inspection setup tanks 11, which are arranged in an interior of the testing device 1 to each receive a reagent disc 2 to set therein, wherein the reagent disc 2 is formed with a plurality of reaction receptacles 21 for receiving an antibiotic susceptibility testing (AST)-dedicated indicator 211 therein;
a separation plate 12 arranged between the inspection setup tanks 11, a cover 13 movably arranged on each of the inspection setup tanks 11;
at least two culture elements 3, which are respectively arranged at one side of the inspection setup tanks 11 to each receive and hold an original specimen A1;
a growth recognition light source 4, which is arranged at one side of the culture elements 3 to illuminate the original specimen A1, in order to recognize a growth condition thereof;
at least two first inspection light sources 51, which are respectively arranged at one side of the inspection setup tanks 11 to respectively illuminate the reaction receptacles 21;
at least two second inspection light sources 52, of which a wavelength is different from the first inspection light sources 51 and are respectively set at one side of the inspection setup tanks 11 to illuminate the same ones of the reaction receptacles 21 with the first inspection light source 51;
at least one light inspection module 41, which acquires a growth message through a number of the original specimens A1 obtained by illuminating the culture elements 3 with the growth recognition light source 4; and
at least one spectrum processing module 53, which receives a color message obtained with the AST-dedicated indicator 211 by illuminating the reaction receptacles 21 with the first inspection light sources 51 and the second inspection light sources 52 for conversion into a wavelength message.

A method for operating the antibiotic susceptibility testing device 1 according to the present invention includes the following steps:
(a) pre-cultivating specimen: an original specimen A1 is placed in a culture element 3 of a testing device 1 to carry out pre-cultivation;
(b) determining if cultivation completed: a growth recognition light source 4 illuminates the culture element 3, and a light inspection module 41 acquires a growth message of a number of the original specimen A1 after the illumination of the culture element 3 with the growth recognition light source 4 so as to determine if the cultivation is completed, and if the cultivation is completed, the original specimen A1 becomes a usable specimen A2 and the process enters Step (c), and if the cultivation is not completed, Step (b) is repeated;
(c) diluting specimen: the usable specimen A2 is diluted with a culture solution to become an appropriate specimen A3, which is introduced into at least one reaction receptacle 21 of a reagent disc 2;
(d) testing specimen: the reagent disc 2 is placed into at least one inspection setup tank 11 of the testing device 1, and the testing device 1 is activated;
(e) inspecting with dual light sources: a first inspection light source 51 and a second inspection light source 52 having different wavelengths are applied in sequence to illuminate the appropriate specimen A3 of a same one of the at least one reaction receptacle 21; and
(f) judging with spectrum message: a spectrum processing module 53 is operated to receive a color message obtained through illuminating an AST-dedicated indicator 211 of the reaction receptacle 21 for conversion into a wavelength message so as to obtain a result of inspecting.

The testing device 1 is a box structure with an upward-opening cover, of which the cover 13 is two in number and can be opened independently; the inspection setup tank 11 is a tank-like accommodating space in the testing device 1 and has a space for setting up the reagent disc 2 therein, and the culture element 3 is located at one side of the setup area in the testing device 1, and in the instant embodiment, culture bottles located at corners of two inspection setup tanks 11 are provided as an example; the growth recognition light source 4 is a light emitting element with a specified wavelength, and the light inspection module 41 is a light receiver corresponding to the growth recognition light source 4 and has a chip with a function of calculating a growth state, and in the instant embodiment, the growth recognition light source 4 and the light inspection module 41 are arranged on two opposite sidewalls of a setup seat of the culture element 3; the first inspection light source 51 and the second inspection light source 52 are light emitting elements of specified wavelengths, and the spectrum processing module 53 is a light receiver corresponding to the first inspection light source 51 and the second inspection light source 52, and has a chip with a function of converting a color message, and in the instant embodiment, the first inspection light source 51 and the second inspection light source 52 are arranged on the cover 13 of the testing device 1, and illumination directions are controlled so as to illuminate in the same reaction receptacle 21, and in principle, illumination angles of the first inspection light source 51 and the second inspection light source 52 are fixed, so that illumination can be made on different reaction receptacles 21 by rotating the reagent disc 2, and the spectrum processing module 53 is arranged on a bottom of the testing device 1 at a location adjacent to the reaction receptacle 21; or the chips of the light inspection module 41 and the spectrum processing module 53 can be integrated into a single processing chip, which is set on the testing device 1 and electrically connected thereto to distinguish the light receiving function from the calculation function. The corresponding types of the above components are only examples for the preferred embodiment, and all types with the same functions belong to the scope of the present invention and are not limited to the above examples.

The structure of this technology can be understood through the above description, and according to the corresponding combination of such a structure, it is possible achieve the advantages of accurately judging the growth states of the specimen before and after AST test and the result of testing in a simple and fast way. It can be clearly seen from the drawings that before using the present invention to perform antibiotic susceptibility testing, a user first places the original specimen A1 in the culture element 3 inside the testing device 1 for pre-cultivation, and during the cultivation period, the growth recognition light source 4 can be used to illuminate the culture element 3, and after the light inspection module 41 receives the illumination of the growth recognition light source 4, the growth message obtained from the change of the number of the original specimen A1 is used to determine whether it has been cultivated into a usable specimen A2, and in the instant embodiment, the light inspection module 41 performs the inspection by turbidimetry, where turbidimetry is a method for evaluating the concentration of suspended particles in a solution, and therefore, the growth recognition light source 4 is used to emit light through the culture element 3, and the light inspection module 41 measures the light intensity passing through the bacterial liquid, wherein the decrease in light intensity is proportional to the concentration of the specimen in the bacterial liquid, and the concentration of the bacterial liquid can be standardized using a McFarland turbidimetric standard to ensure that the concentration of the bacterial liquid is consistent between different experiments, or a standard curve of known specimen concentration can be established so that the inspection data can be compared with the standard curve to determine the concentration of the specimen in the bacterial liquid to ensure that the specimen is growing stably and has a sufficient concentration before testing, so as to avoid invalid testing.

Then, the usable specimen A2 is diluted with the culture solution to become the appropriate sample A3, and added to the reaction receptacle 21 of the reagent disc 2, and then placed in the inspection setup tank 11 of the testing device 1 to then activate the testing device 1, and in order to make the reaction conditions of each reaction receptacle 21 the same, in addition to setting an equal amount of AST-dedicated indicator 211 in the reaction receptacle 21, a centrifugal force is applied to have the appropriate specimen A3 evenly distributed to each reaction receptacle 21, and the appropriate specimen A3 being diluted is more conducive to the above operation of distribution, and the dilution of the usable specimen A2 can also make the baseline of the bacterial liquid concentration consistent, so that the concentration of the appropriate specimen A3 before the reaction can be more accurately controlled.

After the reaction is completed, the testing device 1 automatically applies the first inspection light source 51 and the second inspection light source 52 of different wavelengths to sequentially illuminate the appropriate specimen A3 in the same reaction receptacle 21, and the reaction of the appropriate specimen A3 with the AST-dedicated indicator 211 generates a color change, and the color change is different for different AST-dedicated indicator 211 used, and for example, when the AST-dedicated indicator 211 used is a resazurin indicator, three cover changes for blue, purple, and pink will be generated, and when the AST-dedicated indicator 211 used is 2,3,5-triphenyltetrazolium chloride indicator, color changes for colorlessness, light pink, and dark pink are generated, and in the instant embodiment, the resazurin indicator is taken as an example for the AST-dedicated indicator 211, wherein the resazurin indicator is an oxidation-reduction indicator, which is blue in color, so that the color displayed is blue before the specimen does not yet grow, and will be reduced to purple or pink upon being acted by NADH dehydrogenase of specimen mitochondria, wherein NAD is nicotinamide adenine dinucleotide, which is a coenzyme of dehydrogenase, and NADH is a reduced state of NAD. Therefore, the color before the reaction is detected by the first inspection light source 51 with an absorbance value of OD 600-580 nm, and the color after the reaction is detected by the second inspection light source 52 with an absorbance value of OD 580-560 nm, and if the AST-dedicated indicator 211 is 2,3,5-triphenyltetrazolium chloride indicator, detection is made with an absorbance value of OD 245-248 nm.

After illuminating the AST-dedicated indicator 211, the first inspection light source 51 and the second inspection light source 52 pass through the reaction receptacle 21 to reach the spectrum processing module 53, and the spectrum processing module 53 receives the message information after the illumination and converts it into a wavelength message to thereby obtain the result of inspection, wherein the result of inspection is no longer a general color, but a specific number, so that by comparing the wavelength message before and after the reaction, it is easy to eliminate abnormal data or select the data with the most significant result to serve as a judgement standard. As the color change of the AST-dedicated indicator 211 is gradual, the wavelength message converted from the color message is actually a spectral range, and as shown in Table 1, based on testing of the present invention, the best inspection wavelengths are respectively wavelength of the first inspection light source 51 being 600 nm and the wavelength of the second inspection light source 52 being 570 nm. For example, the wavelength messages of four groups of bacterial liquids before and after the reaction are respectively (0.928, 0.198), (0.666, 0.43), (0.535, 0.566), and (0.377, 0.523). It can learn from this that the color change determined by humans may have errors, and the closer the data before and after the reaction, the more significant the antibacterial effect of the antibiotic. In this way, the growth state of the specimen and the results of testing can be determined accurately in a simple and fast manner, and the results of test are analyzed with two inspection light sources and the spectrum processing module 53, which not only avoids manual interpretation, but also can obtain more accurate results through cross-comparison.

TABLE 1

| Color before reaction | Blue | Blue | Blue | Blue |
|---|---|---|---|---|
| Color after reaction | Red | Purple | Dark Purple | Blue |
| Value of OD 570 | 0.928 | 0.666 | 0.535 | 0.377 |
| Value of OD 600 | 0.198 | 0.43 | 0.566 | 0.523 |
| Color Judged | Red | Purple | Purple | Blue |
| Spectral Range | 480-600 | 480-640 | 500-640 | 500-640 |

Further, the testing device 1 of the present invention has two inspection setup tanks 11, and the two inspection setup tanks 11 are provided with a separation plate 12 arranged therebetween and are each provided with a cover 13, so that the two inspection setup tanks 11 can operate independently, and the separation plate 12 can separate the two inspection setup tanks 11 to prevent specimens from being infected with each other. In addition to the effects of being dust-proof and anti-contamination, the cover 13 can also remind a user of the working status of the testing device 1, such as setting up a safety mechanism by allowing activation only when the cover 13 is closed. As such, the operation of the testing device 1 is made more convenient and more efficient.

Further, the testing device 1 of the present invention is provided with a high-speed driving module 54 to rotate each reagent disc 2 to have gas bubbles generated by the reaction in each reaction receptacle 21 driven away from the center position before each first inspection light source 51 and each second inspection light source 52 are activated. The high-speed driving module 54 is a control software-loaded chip (such as being at one side of the spectrum processing module 53), or can be integrated into the chip of the spectrum processing module 53, and can be used in combination with a timer and a rotating motor in the testing device 1 for practice. Since the specimen and the AST-dedicated indicator 211 need to be left to react for a period of time after being mixed, and gas bubbles will be generated during the process of biochemical reaction, the bubbles may cause the light of the first inspection light source 51 and the second inspection light source 52 to shift so as to affect the signal reading operation of the spectrum processing module 53. Therefore, after the time of being left still is over and before the first inspection light source 51 and the second inspection light source 52 are put into operation, the high-speed driving module 54 in the testing device 1 is used to rotate the reagent disc 2 at a high speed for a short period of time, so as to apply a centrifugal force to drive the bubbles in the reaction receptacle 21 away from the center position, thereby ensuring that the spectrum processing module 53 can receive light source signals correctly and stably.

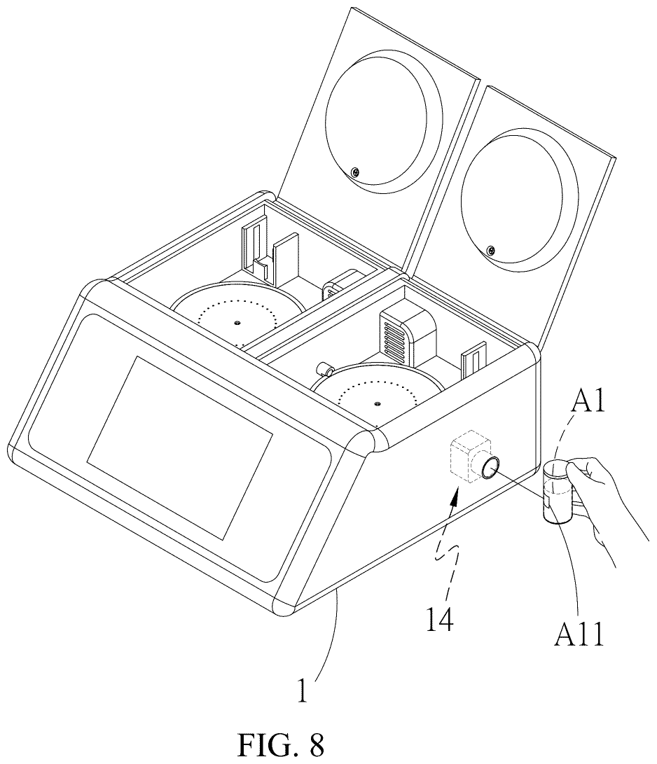

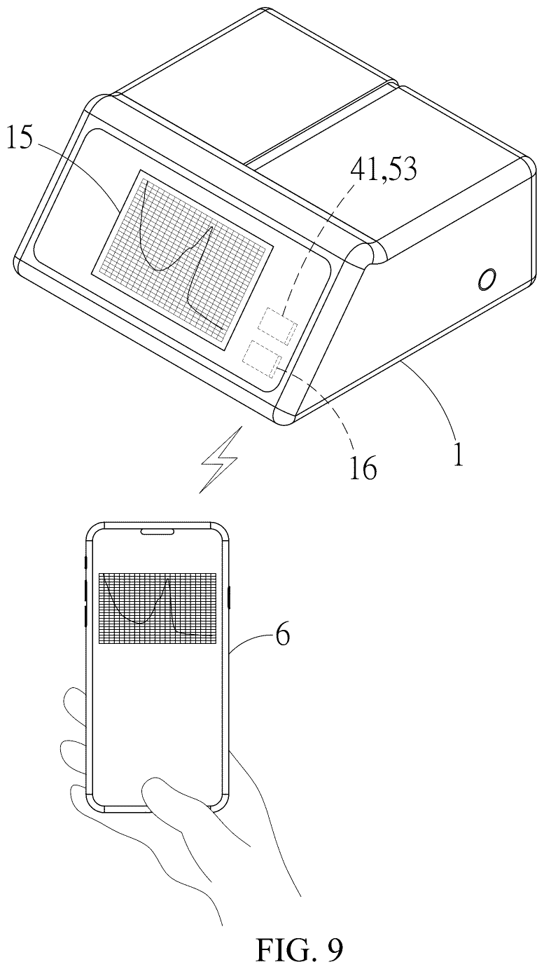

Referring to FIGS. 8 and 9, which are respectively a schematic view illustrating specimen recognizing according to another preferred embodiment of the present invention and a schematic view illustrating displaying according to said another preferred embodiment of the present invention, it is clearly seen from the drawings that the instant embodiment is similar to the previous embodiment and is only different in that the testing device 1 is provided with a recognition reading portion 14 on the outside thereof, and in the instant embodiment, the recognition reading portion 14 is a combination of a lens and a recognition module. As such, before the testing device 1 is activated, the recognition reading portion 14 on the outside of the testing device 1 is first used to reads a container recognition portion A11 of the original specimen A1, wherein the recognition module can carry out recognition of the container recognition portion A11 such as text, barcode, or QR code, so that when performing the specimen culture operation, the user can first use the recognition reading portion 14 to confirm whether the disc item is correct, so as to avoid the problem of misjudgment by the user's naked eyes to improve the overall convenience and efficiency of operation. Further, as the recognition operation is performed without contacting the testing, so as to also avoid the recognition operation affecting the operation stability of the testing device 1. Of course, the basis for judgment requires first establishing corresponding data in a database, and this will not be repeated.

Further, the testing device 1 is provided with a display screen 15, which is electrically connected to the spectrum processing module 53, and a network-connecting module 16 is provided in the testing device 1 to transmit the result of inspection from the spectrum processing module 53 to an electronic device 6. In the instant embodiment, the chips of light inspection module 41 and the spectrum processing module 53 are integrated into a processing chip, which is arranged on the testing device 1 and electrically connected thereto. In this way, the color message, the wavelength message, or the result of inspection obtained by the spectrum processing module 53 can be directly displayed on the display screen 15, eliminating the need for the user to connect additional transmission lines or other display devices, allowing for more intuitively learning the results of experiments, and the network-connecting module 16 can also be used to transmit the color message, the wavelength message, or the result of inspection obtained by the spectrum processing module 53 to the electronic device 6, such as a mobile phone, at a remote site, and the content on the display screen 15 can be synchronously displayed on a screen of the electronic device 6.

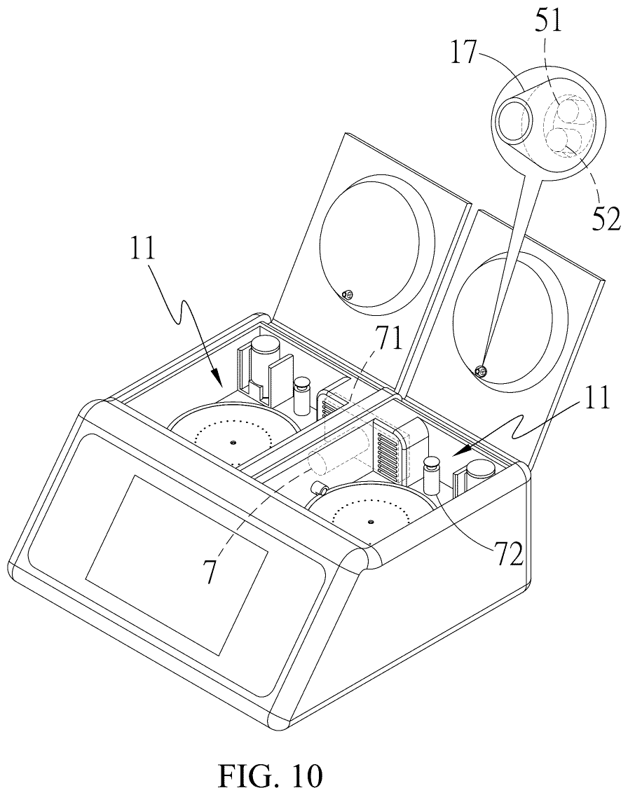

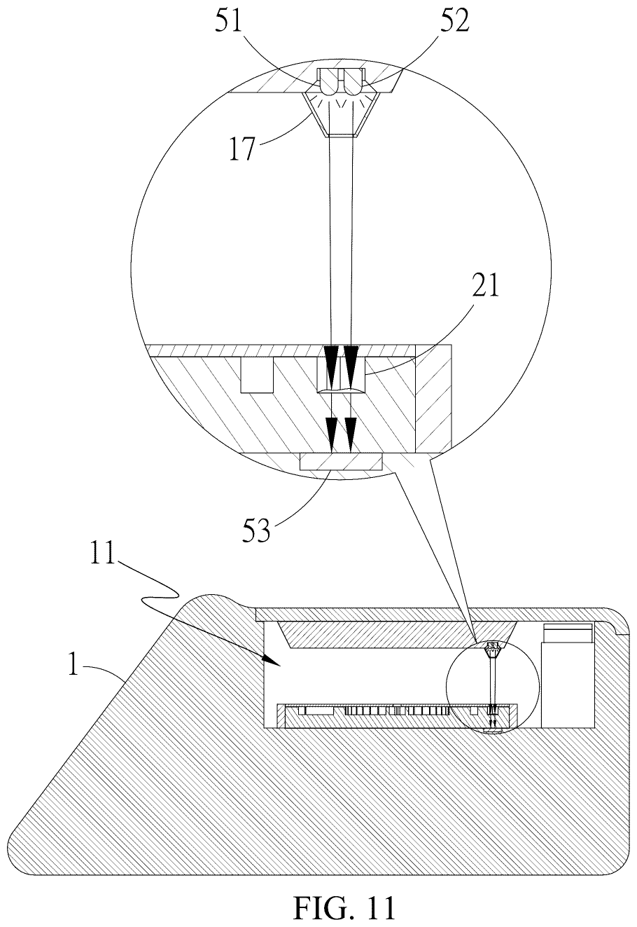

Referring to FIGS. 10 and 11, which are respectively a schematic view illustrating temperature uniformizing according to a further preferred embodiment of the present invention and a schematic view illustrating light converging according to said further preferred embodiment of the present invention, it is clearly seen from the drawings that the instant embodiment is similar to the previous embodiment and is only different in that the testing device 1 is provided with a heating device 7 and a temperature-uniformizing channel 71 arranged at one side of the heating device 7 and in communication with each of inspection setup tank 11 to maintain the temperature inside each inspection setup tank 11 according to a temperature sensor 72 in arranged each inspection setup tank 11. In this way, the two temperature sensors 72 respectively detect the temperatures inside the inspection setup tanks 11, and heating may be carried out by the heating device 7 according to a set temperature. However, when a temperature drop is caused by certain factors (such as the cover at one of the sides is opening), in order to avoid the temperature drop affecting the temperature in the other inspection setup tank 11, as long as the temperature fed back from any one of the temperature sensors 72 is lower than a set value, the testing device 1 automatically activates the heating device 7 to blow out hot air of the set temperature, and the hot air is conveyed to the two inspection setup tanks 11 through the temperature-uniformizing channel 71 in communication with the two inspection setup tanks 11. In this way, the one having a lower temperature can restore the temperature by means of the hot air, while the one having the normal temperature is not affected due to the temperature of the hot air being corresponding thereto, thereby achieving the purpose of maintaining the temperatures of the two inspection setup tanks 11 to be consistent.

Further, a light-converging element 17 is provided at one side of the first inspection light source 51 and the second inspection light source 52 in the same inspection setup tank 11 to have light converging on the reaction receptacle 21. In the instant embodiment, the light-converging element 17 is a hood of a truncated cone, of which an inside wall has an effect of reflecting light, with light only transmittable through a front end. The front end can be hollowed out or provided with a convex lens for converging light, so that converging light beam is fulfilled to make the inspection light source converging and precisely projected into the reaction receptacle 21 of the reagent disc to avoid influence by scattering so as to allow the spectrum processing module 53 to collect data more accurately.

I claim:

1. An apparatus for antibiotic susceptibility testing (AST), comprising:
a testing device;
at least two inspection setup tanks, which are arranged in the testing device to each receive a reagent disc to set therein, wherein the reagent disc is formed with a plurality of reaction receptacles for receiving an AST-dedicated indicator therein;
at least two culture elements, which are respectively arranged at one side of the inspection setup tanks to each receive and hold an original specimen;
a growth recognition light source, which is arranged at one side of the culture elements to illuminate the original specimen, in order to recognize a growth condition thereof;
at least two first inspection light sources, which are respectively arranged at one side of the inspection setup tanks to respectively illuminate the reaction receptacles;
at least two second inspection light sources, of which a wavelength is different from the first inspection light sources and are respectively set at one side of the inspection setup tanks to illuminate same ones of the reaction receptacles as the first inspection light sources;
at least one light inspection module, which acquires a growth message through a number of the original specimens obtained by illuminating the culture elements with the growth recognition light source; and
at least one spectrum processing module, which receives a color message obtained with the indicator by illuminating the reaction receptacles with the first inspection light sources and the second inspection light sources for conversion into a wavelength message.

2. The apparatus for AST according to claim 1, wherein a recognition reading portion is arranged on an outside of the testing device, and a display screen is provided on the testing device and electrically connected to the spectrum processing module, and a network-connecting module is arranged inside the testing device to transmit a result of inspection from the spectrum processing module to an electronic device.

3. The apparatus for AST according to claim 1, wherein the testing device is provided with a heating device and a temperature-uniformizing channel in communication with each of the inspection setup tanks and maintains a temperature in each of the inspection setup tanks according to a temperature sensor arranged in each of the inspection setup tanks.

4. The apparatus for AST according to claim 1, wherein a light-converging element is arranged at one side of the first inspection light source and the second inspection light source of a same one of the inspection setup tanks to have light converging on the reaction receptacle.

5. The apparatus for AST according to claim 1, wherein the testing device is provided with a high-speed driving module to rotate each of the reagent discs to drive gas bubbles generated by reaction occurring in each of the reaction receptacles away from a center position before each of the first inspection light sources and each of the second inspection light sources are activated.

6. A method for operating a testing device of antibiotic susceptibility testing (AST), comprising the following steps:
(a) placing an original specimen in a culture element in a testing device to carry out pre-cultivation;
(b) applying a growth recognition light source to illuminate the culture element, and determining if the cultivation is completed by having a light inspection module acquiring a growth message through number of the original specimens after the illumination of the culture element with the growth recognition light source, and if the cultivation is completed, the original specimen becoming a usable specimen, and proceeding to Step (c), and if the cultivation is not completed, repeating Step (b);
(c) diluting the usable specimen with a culture solution into an appropriate specimen to be introduced into at least one reaction receptacle of a reagent disc;
(d) placing the reagent disc into at least one inspection setup tank of the testing and activating the testing device;
(e) sequentially applying a first inspection light source and a second inspection light source having different wavelengths to illuminate the appropriate specimen of a same one of the at least one reaction receptacle; and
(f) applying a spectrum processing module to receive a color message obtained through illuminating an AST indicator in the reaction receptacle for conversion into a wavelength message, so as to obtain a result of inspection.

7. The method for operating the testing device of AST according to claim 6,
wherein after Step (d), Step (d1) is performed, in which before the activation of the testing device, a recognition reading portion on an outside of the testing device is first used to read a container recognition portion of the usable specimen, and after Step (f), Step (f1) is performed, in which the result of inspection is displayed on a display screen of the testing device, and after Step (f1), Step (f2) is performed, in which the result of inspection is transmitted through a network-connecting module to an electronic device.

8. The method for operating the testing device of AST according to claim 6, wherein after Step (a), Step (a1) is performed, in which a temperature sensor is operated to detect a temperature inside the inspection setup tank, and through a temperature-uniformizing channel in communication with the inspection setup tank, the temperature inside the inspection setup tank is kept by means of a heating device.

9. The method for operating the testing device of AST according to claim 6, wherein a light-converging element is arranged at one side of the first inspection light source and the second inspection light source to have light converging on the reaction receptacle.

10. The method for operating the testing device of AST according to claim 6, wherein in Step (e), before the first inspection light source and the second inspection light source are activated, a high-speed driving module in the testing device is operated to rotate the reagent disc so as to drive gas bubbles generated through reaction in the reaction receptacle away from a center position.
